(19) United States
(12) Patent Application Publication
(10) Pub. No.: US 2009/0209858 A1
(43) Pub. Date: Aug. 20, 2009

(54) SYSTEM AND METHOD FOR ULTRASONIC IMAGE PROCESSING

(75) Inventor: MICHAEL L. OELZE, Champaign, IL (US)

Correspondence Address: AKERMAN SENTERFITT, P.O. BOX 3188, WEST PALM BEACH, FL, 33402-3188, US

(73) Assignee: THE BOARD OF TRUSTEES OF THE UNIVERSITY OF ILLINOIS, Urbana, IL (US)

(21) Appl. No.: 12/370,512

(22) Filed: Feb. 12, 2009

Related U.S. Application Data
(60) Provisional application No. 61/029,479, filed on Feb. 18, 2008.

(51) Int. Cl. A61B 8/00

(52) U.S. Cl. 600/443

(57) ABSTRACT

A system that incorporates teachings of the present disclosure may include, for example, an ultrasonic (US) imaging system having a transducer, and a controller managing operations of the transducer. The controller can be operative to cause the transducer to transmit a coded ultrasound signal to an object, cause the transducer to receive an altered coded ultrasound signal from the object, decode the altered coded ultrasound signal to attain a desired bandwidth that exceeds an impulse response of the US imaging system, subdivide the attained bandwidth of the decoded ultrasound signal into a plurality of sub-bands, and generate an image for each sub-band from signals included in each sub-band of the decoded ultrasound signal. Additional embodiments are disclosed.

### CROSS REFERENCE TO RELATED APPLICATIONS

The present application claims the benefit of priority to U.S. Provisional Patent Application, Ser. No. 61/029,479, filed Feb. 18, 2008, by Dr. Michael L. Oelze, entitled “Ultrasonic Imaging Speckle Suppression and Contrast Enhancement Technique,” which is hereby incorporated by reference in its entirety.

### STATEMENT AS TO FEDERALLY SPONSORED RESEARCH

This invention was made with government support under the National Institute of Health awarded under R21 EB006741. The government has certain rights in this invention.

### FIELD OF THE DISCLOSURE

The present disclosure relates generally to ultrasonic image processing, and more specifically to a system and method for ultrasonic image processing.

### BACKGROUND

Speckle, the granular structure in ultrasonic images, reduces the ability to detect low-contrast targets. Speckle is formed by subresolution scatterers that cause constructive and destructive interference of backscattered ultrasonic signals within the resolution cell volume of an ultrasonic source. Furthermore, speckle is considered to be a deterministic process because when an object is imaged under the same operating conditions no changes in the speckle pattern occur. It is because of this nature, speckle is not reduced by signal averaging. Therefore, a considerable amount of work and effort has been spent over the last few decades in developing techniques to reduce speckle in ultrasound images.

Speckle reduction techniques can be classified into two categories: post-processing techniques and compounding methods. Examples of some of the post-processing techniques are adaptive filtering (linear and non-linear), deconvolution, and wavelet despeckling. The compounding speckle reduction methods include spatial and frequency compounding. These schemes rely on making separate images that have uncorrelated or partially correlated speckle patterns and then are averaged to reduce the speckle but at the expense of spatial resolution. Originally for spatial compounding, the source aperture was translated laterally or at different angles to make images from different orientations.

The main drawbacks of these techniques are loss in lateral resolution, and image alignment due to motion which causes image artifacts. Also, the need for multiple images would mean a decrease in the frame rate. However, recently manufacturers have employed receive aperture-only spatial compounding which is not subject to frame rate losses or motion-based image registration errors. Other advances in spatial-compounding use electronic-beam steering to obtain images at different angles to overcome some of these tradeoffs by using advanced image registration.

Another method, known as frequency compounding (FC), can be applied on transmit mode by using multiple sources at different frequencies or on receive mode as a post-processing speckle reduction technique by dividing the spectrum of the radio-frequency (RF) echoes into subbands to make separate images[1-7]. The latter instance is also known as frequency diversity[2,7], or split spectrum processing[5]. The main disadvantage introduced by using frequency compounding is the inherent tradeoff between axial and contrast resolution. Consequently, if the axial resolution and the bandwidth of an ultrasonic imaging system could be increased these tradeoffs between axial and contrast resolution could be extended.

The first researchers that applied FC to medical ultrasound were Abbott and Thurstone[1]. In this study, they compared laser speckle to ultrasound speckle and suggested several techniques to generate independent speckle patterns including FC. Magnin et al.[2] observed that decorrelation of speckle patterns is dependent upon the excitation bandwidth and obtained increases in speckle signal-to-noise ratio (sSNR) of 26% in B-mode images using a phased array system. Melton et al.[4] developed a model to predict the amount of speckle reduction in A-mode scans based upon correlation coefficients. Trahey et al.[6] discussed a method for optimal speckle reduction based on the number of images that should be acquired within the available system bandwidth. Gehlbach et al.[2] studied frequency diversity using digital filtering techniques to determine a method to maximize sSNR and increase the ability to detect low-contrast targets. In addition, it was observed that a 10 to 15 percent increase in sSNR can be achieved by increasing the number of filters and filter overlap. Stetson et al. [8] used frequency diversity techniques with the combination of gray level mapping to improve the contrast-to-noise ratio (CNR) of low-contrast targets.

A coded excitation and pulse compression technique was recently developed, resolution enhancement compression (REC), which allows the axial resolution and bandwidth of the imaging system to be enhanced[9]. In addition to improvements in terms of axial resolution, the REC technique has the typical coded excitation and pulse compression benefits, such as deeper penetration due to improvement in echo signal-to-noise ratio (eSNR).

### BRIEF DESCRIPTION OF THE DRAWINGS

FIG. 1 depicts an illustrative embodiment of a convolution equivalence from simulations: a) pulse-echo impulse response with a 50%—3-dB bandwidth, b) pre-enhanced chirp used to excite the 50% source, c) convolution of 50% source with pre-enhanced chirp, d) pulse-echo impulse response with a 100%—3-dB bandwidth, e) linear chirp used to excite the 100% source f) convolution of 100% source with linear chirp;

FIG. 2 depicts an illustrative embodiment of a) simulated envelope from a point scatterer in an attenuated media, and b) PSD for CP and the REC compressed waveforms;

FIG. 3 depicts an illustrative embodiment of a block diagram of a FC scenario with three subbands;

FIG. 4 depicts an illustrative embodiment of the bandwidth for all four FC cases applied in conjunction with the bandwidth of the reference signals for CP and REC, wherein the REC center frequency was shifted to eliminate any dc-component in the spectrum;

FIG. 5 depicts an illustrative embodiment of a) Normalized CNR vs pulse length increase factor, b) Normalized sSNR_{B} vs pulse length increase factor, c) Normalized sSNR_{T} vs pulse length increase factor, d) Normalized lSNR vs pulse length increase factor. For simulations and experiments, the quality metric values are normalized to the data corresponding to conventional pulsing with a pulse length increase factor of one. (simulated results are depicted by a solid line, experimental measurements are shown in a dashed line, REC results are marked by diamonds, and CP results are marked by circles);

FIGS. 6A-6E depict illustrative embodiments of B-mode images of simulated results for: a) CP and REC reference scans, b) REC-FC full-width case, c) CP-FC and REC-FC half-width cases, d) CP-FC and REC-FC third-width cases, and e) CP-FC and REC-FC fourth-width cases. Image dynamic range=−50 dB;

FIG. 7 depicts an illustrative embodiment of edge detection images of simulated results using thresholding and margin strength for CP, REC and REC-FC full-width case;

FIGS. 8A-8E depict illustrative embodiments of histograms of simulated results for: a) CP and REC reference scans, b) REC-FC full-width case, c) CP-FC and REC-FC half-width cases, d) CP-FC and REC-FC third-width cases, and e) CP-FC and REC-FC fourth-width cases. (background region,—target region);

FIGS. 9A-9B depict illustrative embodiments of a) Block diagram of experimental setup for CP, and b) block diagram of experimental setup for REC;

FIGS. 10A-10E depict illustrative embodiments of B-mode images of experimental measurements for: a) CP and REC reference scans, b) REC-FC full-width case, c) CP-FC and REC-FC half-width cases, d) CP-FC and REC-FC third-width cases, and e) CP-FC and regions for all four cases are shown in FIG. 12. REC-FC fourth-width cases. Image dynamic range=−50 dB;

FIGS. 11A-11E depict illustrative embodiments of histograms of experimental measurements for: a) CP and REC reference scans, b) REC-FC full-width case, c) CP-FC and REC-FC half-width cases, d) CP-FC and REC-FC third-width cases, and e) CP-FC and REC-FC fourth-width cases. ( [symbol] background region,—target region);

FIG. 12 depicts an illustrative diagrammatic representation of a machine in the form of a computer system within which a set of instructions, when executed, may cause the machine to perform any one or more of the methodologies disclosed herein;

TABLE 1 depicts an illustrative embodiment of Filter Bank Descriptions, Axial and Lateral Correlation Functions for the 50 Cases of Simulated RF Data for a 15 mm target. The Resolution Values In The Table Are Described In Terms Of The Mean Plus/Minus One Standard Deviation. Values In Parentheses Represent The Absolute Percent Change Of Rec-Fc Vs. Cp Reference;

TABLE 2 depicts an illustrative embodiment of a CNR, sSNR, HPI, HO and LSNR for the 50 cases of simulated RF data for a 15 mm target. The values in the table are described in terms of the mean plus/minus one standard deviation. In parenthesis: absolute percent change of REC-FC vs. CP reference—absolute percent change of REC-FC vs. CP-FC for the same case;

TABLE 3 depicts an illustrative embodiment of a filter bank descriptions, axial and lateral correlation functions for the experimental results for the 15 mm target from the ATS phantom. Values in parentheses represent the absolute percent change of REC-FC vs. CP reference; and

TABLE 4 depicts an illustrative embodiment of a CNR, sSNR, HPI, and LSNR for the experimental results for the 15 mm target from the ATS phantom. The HPI values in the table are described in terms of the mean plus/minus one standard deviation. In parenthesis: absolute percent change of REC-FC vs. CP reference—absolute percent change of REC-FC vs. CP-FC for the same case.

### DETAILED DESCRIPTION

One embodiment of the present disclosure entails transmitting a coded ultrasound signal to an object, receiving an altered coded ultrasound signal from the object, decoding the altered coded ultrasound signal to attain a desired bandwidth that is larger than a bandwidth of an impulse response of an imaging system, subdividing the attained bandwidth of the decoded ultrasound signal into a plurality of sub-bands, and generating an image for each sub-band from signals included in each sub-band of the decoded ultrasound signal.

Another embodiment of the present disclosure entails an ultrasonic (US) imaging system having a transducer, and a controller managing operations of the transducer. The controller can be operative to cause the transducer to transmit a coded ultrasound signal to an object, cause the transducer to receive an altered coded ultrasound signal from the object, decode the altered coded ultrasound signal to attain a desired bandwidth that exceeds an impulse response of the US imaging system, subdivide the attained bandwidth of the decoded ultrasound signal into a plurality of sub-bands, and generate an image for each sub-band from signals included in each sub-band of the decoded ultrasound signal.

Yet another embodiment of the present disclosure entails computer-readable storage medium having computer instructions to decode a coded ultrasound signal to attain a desired bandwidth, subdivide the attained bandwidth of the decoded ultrasound signal into a plurality of sub-bands, and generate an image for each sub-band from signals included in each sub-band of the decoded ultrasound signal.

Another embodiment of the present disclosure entails a computer-readable storage medium having computer instructions to generate ultrasound images from a coded ultrasound signal processed according to a combination of a speckle reduction technique of frequency compounding and a coded excitation and pulse compression technique.

Another embodiment of the present disclosure entails combining a speckle reduction technique of frequency compounding and a coded excitation and pulse compression technique to generate ultrasound images.

Yet another embodiment of the present disclosure entails a method involving scanning an object with a Ultrasonic Imaging system operative to transmit a coded ultrasound signal to the object, receive an altered coded ultrasound signal from the object, decode the altered coded ultrasound signal to attain a desired bandwidth, subdivide the attained bandwidth of the decoded ultrasound signal into a plurality of sub-bands, and generate an image for each sub-band from signals included in each sub-band of the decoded ultrasound signal.

Yet another embodiment of the present disclosure entails scanning an object with an Ultrasonic Imaging system that combines a speckle reduction technique of frequency compounding and a coded excitation and pulse compression technique to generate ultrasound images.

Yet another embodiment of the present disclosure entails diagnosing a patient scanned with an Ultrasonic Imaging system that combines a speckle reduction technique of frequency compounding and a coded excitation and pulse compression technique to generate ultrasound images.

Yet another embodiment of the present disclosure entails an Ultrasonic Imaging system having a controller to generate ultrasound images by combining a speckle reduction technique of frequency compounding and a coded excitation and pulse compression technique.

The driving force behind the REC technique[9] is the capability to shape and select to a limited degree certain desired characteristics of an ultrasonic imaging system through coded excitation and pulse compression. Consequently, the characteristics of the impulse response of the imaging system could be tailored to have useful properties for particular imaging applications. For example, if the useable bandwidth of the imaging system could be increased using REC, the increase in bandwidth could be used with FC to improve target contrast while retaining the original axial resolution of the imaging system.

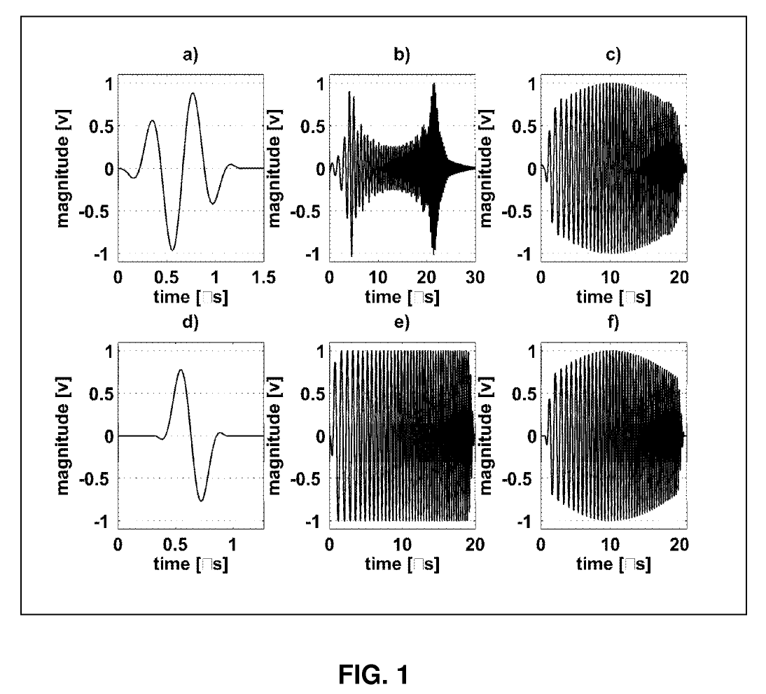

In REC, a pre-enhanced chirp is used to selectively excite an ultrasonic source with different energies at chosen frequencies. Using the concept of convolution equivalence in the frequency domain as described in[9], a pre-enhanced chirp can be found by applying the following equation,

$${V}_{pre} = \frac{{H}_{1}^{*} ( f , x )}{{\vert {H}_{1} ( f , x ) \vert}^{2} + {\vert {H}_{1} ( f , x ) \vert}^{- 2}} \cdot {H}_{2} ( f , x ) \cdot {V}_{Lin} ( f ) \qquad ( 1 )$$

where H₁(f, x) is the spatially varying Fourier spectrum of the pulse-echo impulse response, H₂(f, x) is the spatially varying Fourier spectrum of the desired response, and V_{Lin}(f) is the Fourier spectrum of a linear chirp. A time-domain example of the convolution equivalence is illustrated in FIG. 1. By exciting the transducer with the pre-enhanced chirp, the bandwidth is enhanced due to the increase of energy in the frequency bands that normally would be filtered in some measure by the bandpass nature of the transducer. Conceptually, to obtain a constant eSNR per frequency channel across the desired bandwidth, the additional amount of energy required on transmit at the outer frequency bands will depend on the original transducer's bandwidth and the amount of bandwidth boost desired. For example, if a source with a −6-dB bandwidth of 50% is boosted 100%, up to and more than twice as much energy can be pumped into the inefficient bands compared to the center frequency of the source.

After exciting the source with a pre-enhanced chirp, the received signal is compressed using a Wiener filter based on convolution equivalence. The resulting backscattered signal has an impulse response h₂ (nT, x). Wiener filtering is described by the following equation:

$${β}_{REC} ( f ) = \frac{{V}_{Lin}^{′ *} ( f )}{{\vert {V}_{Lin}^{′} ( f ) \vert}^{2} + γ {\overline{e S N R}}^{- 1} ( f , x )} \qquad ( 2 )$$

where V′_{Lin}(f) is the Fourier spectrum of a modified linear chirp, γ is a smoothing parameter that allows tradeoff between axial resolution, gain in eSNR, and sidelobe levels. A modified linear chirp is used to restore convolution equivalence as the signal is slightly altered and filtered by electronics. e̅S̅N̅R̅[10] is the average eSNR per frequency channel and is defined as:

$$\overline{e S N R} ( f \vert x ) = \frac{{\vert {H}_{2 c} ( f \vert x ) \vert}^{2} E {\{ {\vert F ( f ) \vert}^{2} \}}_{f}}{E {\{ {\vert η ( f ) \vert}^{2} \}}_{e}} \qquad ( 3 )$$

where |F(f)|² is the power spectral density (PSD) of the object function, |η(f)|² is the PSD of the noise, and |H_{2c}(f|x)|² is the PSD of the ensemble average of the compressed signal over noise, h_{2c}(nT|x), which is defined as

h_{2c}(nT,x)=E{g[n]}ₙₒᵢₛₑ (4)

where E{ } is the expected value of the argument and g[n]ₙₒᵢₛₑ is the compressed signal over noise.

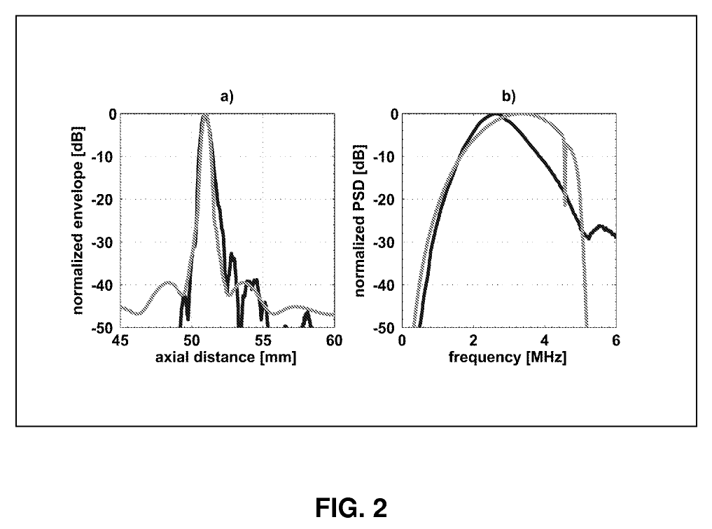

The envelope of the REC waveform (impulse response with double bandwidth) reflected from a point scatterer in an attenuated media (0.5 dB/cm/MHz) at an axial distance of 50 mm and the envelope for conventional pulsing (CP) methods are shown in FIG. 2a. The PSD of REC waveform and CP methods are shown in FIG. 2b to illustrate the bandwidth enhancement that was achieved by using the REC technique.

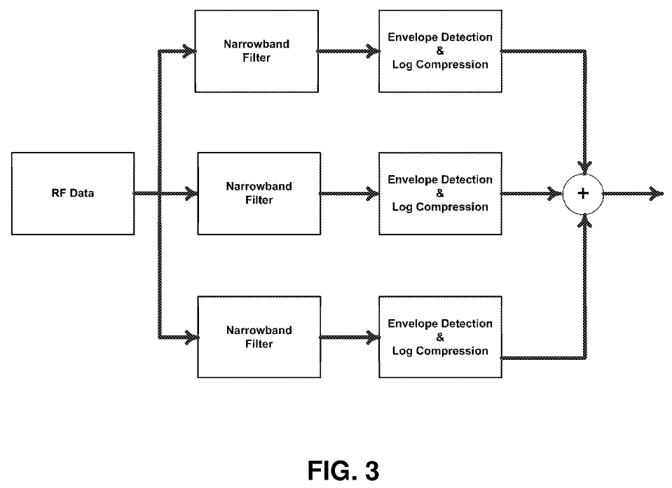

The objective of using FC is to reduce the speckle noise and enhance the contrast in ultrasonic B-mode images. In FC, the received wideband RF spectrum is partitioned into N subbands by using Gaussian band-pass filters of smaller bandwidth than the original spectrum. These narrowband subbands create separate images that make partially uncorrelated speckle patterns, up to 60% decorrelation[3]. Typically, improvements in sSNR and CNR are proportional to \sqrt{N}; however, because these separate images are partially correlated the improvements are going to be proportional to a factor less than the square root of the sum of uncorrelated images. These separate images can then be added together to reduce the speckle by reducing the image intensity variance. However, the axial resolution deteriorates because the compounded image was generated by averaging smaller subband images. A block diagram shown in FIG. 3 depicts the operation of FC for a system of N=3.

By using REC, a larger bandwidth is available, which would allow an increase in the number of subbands that can be applied for a particular desired axial resolution. Accordingly, a parameter of interest would be the bandwidth of these subbands. Therefore, in simulations and experiments four cases were evaluated in terms of the subband bandwidth when applying FC to the REC technique. The first case consisted of using subbands that are full-width of the true impulse response bandwidth. This will gauge the true benefits of utilizing the REC-FC technique as the resolution of the compounded image will be the same of that for CP methods and the contrast resolution will improve due to FC. Other cases will consist of using subbands smaller than the full-width, specifically, half-, third- and fourth-width of impulse response bandwidth.

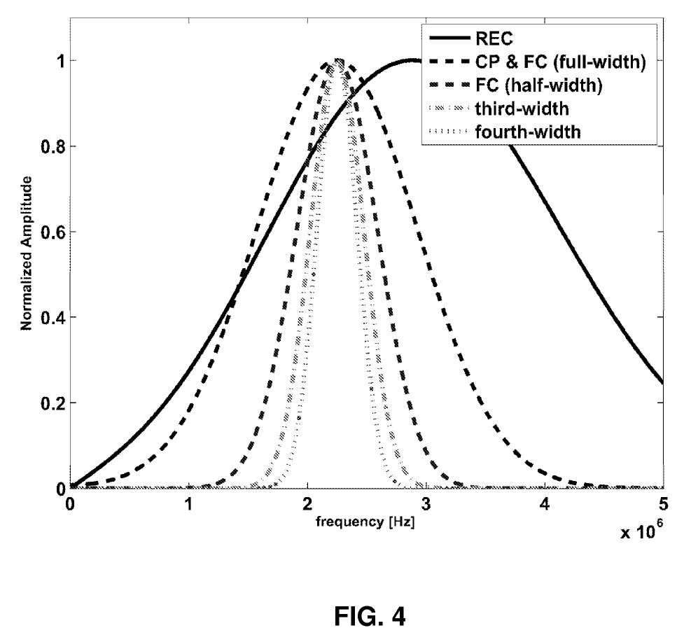

A plot showcasing the size of the subbands for all cases in addition to the original bandwidths of CP and REC is shown in FIG. 4. The cases of genuine interest are full- and half-width as the intention of this work is to improve the contrast without suffering a large amount of resolution loss. However, for study completion the later two cases third- and fourth-width were also evaluated. In addition, for the last three cases, FC will be applied to CP (CP-FC) for comparison purposes. Other filter bank parameters that were considered when applying FC to the RF spectrum were subband center frequency separation, first subband center frequency starting point, and the number of subbands. The values of these parameters were chosen that optimize the image quality metrics, which are defined in the following paragraphs.

To evaluate the performance of the REC-FC technique compared to CP and CP-FC the following image quality metrics were used.

1. Contrast-to-noise ratio (CNR): CNR, also known as contrast-to-speckle ratio, is a quantitative measure that will assess image quality and describe the ability to perceive a target from the background region. CNR is defined as

$$C N R = \vert \frac{{μ}_{B} - {μ}_{T}}{\sqrt{{σ}_{B} + {σ}_{T}}} \vert \qquad ( 5 )$$

where μ_{B} and μ_{T} are the mean brightness of the background and the target lesion and σ_{B} and σ_{T} are the variance of the background and target, respectively. To avoid possible errors in the calculations due to attenuation, the evaluated regions of interest in the background and the target lesion will be of the same size and are located at the same depth.

2. Speckle signal-to-noise ratio (sSNR): sSNR is a measure of the fluctuations in the speckle of a particular region of interest and is defined as

$$s S N R = \frac{μ}{σ} \qquad ( 6 )$$

where μ and σ are the mean and the standard deviation of the region of interest, respectively. Specifically, sSNR will be evaluated for same-sized regions in the target lesion and the background adjacent to the target. For Rayleigh statistics, sSNR is equal to 1.91.

3. Histogram pixel intensity (HPI): HPI is the mean of the frequency distribution of grayscale pixel intensities and is described by

HPI=E{B} (7)

B is the histogram being evaluated and is described by

B(i)=cᵢ (8)

where cᵢ represents the number of pixels in the image within a particular intensity level, i, which is an integer between 0 and 255 that represents the grayscale levels used in B-mode images. Histograms will be made for same-sized regions for the target lesion and the background adjacent to the target. Ideally, for superior target detectability, there is no overlap present between the target histogram and the background histogram. Therefore, histogram overlap (HO) the percentage of overlapping pixels between these two regions will be considered as well.

4. Lesion signal to noise ratio (lSNR): lSNR is a ratio of contrast-detail and resolution. Contrast detail is an analytical measurement of image quality that quantifies the ability of the observer/imaging device to detect an isolated object of minimum size at a fixed contrast, at a given level of observer confidence and for a given noise level. The lSNR relation is defined as:

$$l S N R = \frac{c \cdot d \cdot \sqrt{N}}{\sqrt{{s}_{cx} \cdot {s}_{cz}}} \qquad ( 9 )$$

where d is the diameter of the target lesion, N is the number of uncorrelated images generated with FC (N=1 otherwise), s_{cx} and s_{cz} are the average cell size in the lateral and axial direction, respectively, and c is the contrast of the target and is defined by

$$c = \frac{{ψ}_{1} - {ψ}_{2}}{\sqrt{{ψ}_{1} + {ψ}_{2}}} \qquad ( 10 )$$

where ψ₁ and ψ₂ are the mean-square scattering strength (backscatter intensity) of the background and the target lesion, respectively. The average cell size is obtained using the normalized autocovariance function. It is noteworthy to state that although the correlation function can be evaluated for the compounded image, the results generated are inaccurate because the uncorrelated speckle patterns are averaged, which would morph the speckle size. As a result, the average correlation function was evaluated by using one of the subband images generated.

Computer simulations were carried out in Matlab (Mathworks, Natick, Mass.) to characterize the performance of the REC-FC technique. The simulations used a received pulse-echo pressure field model described as

g′[n]=h₁(nT,x)*f(x)*hₚₑ(nT,y) (11)

where h₁(nT,x) is the pulse-echo impulse response of the transducer, f(x) is the scattering function, and hₚₑ(nT, y) is the modified pulse-echo spatial impulse response that takes into consideration the geometry of the transducer to the spatial extent of the scattered field (beam diffraction). The pulse-echo impulse response, h₁(nT,x), for CP was generated by gating a sinusoid of four cycles with a Hanning window

$$w [ n ] = \{ \begin{matrix}0.5 \cdot [ 1 - cos ( \frac{2 π n}{{L}_{H} - 1} ) ] , & 0 \leq n \leq {L}_{H} - 1 \\ 0 , & otherwise\end{matrix} \qquad ( 12 )$$

where n is an integer and L_{II} is the number of samples in the window. The window and sinusoid parameters where chosen such that it matches the transducer used in experiments. As a result, the pulse-echo impulse response generated is located at the focus of a 2.25 MHz single-element transducer (f/2.66) with a fractional bandwidth of 50% at −3-dB, which would correspond to a window length of N=128. For REC, the desired impulse response function, h₂(nT|x), was constructed to have double the fractional bandwidth or 100% at −3-dB, compared to CP method; therefore, a Hanning window of size of half the length, N=64, was used. The spatial response for a circular focused piston source can be simulated as a circular Gaussian beam which is defined as

$${h}_{pe} ( nT , y ) = {e}^{\frac{- {y}^{2}}{2 {σ}_{y}^{2}}} \qquad ( 13 )$$

where y represents the lateral spatial coordinate and σ_{y}, which is equal to 1.28 mm, is the nominal lateral beamwidth of the source.

The received RF backscatter data were sampled at a rate of 100 MHz and the transducer was translated laterally in increments of 0.1 mm. The received RF data have a size of 4096×300. The object being imaged was a 30 mm×30 mm×1.92 mm simulated phantom. A cylindrical target with a radius of 7.5 mm was located at the center of the phantom. To generate a hyperechoic target with a contrast of approximately +6 dB, the density of point scatterers for the cylindrical volume was four times the density of the remaining volume of the phantom, which will be described throughout as the background. Specifically, to generate fully developed speckle, the cylinder had an average of 20 point scatterers per resolution cell volume and the background had an average of five point scatterers per resolution cell volume. The point scatterers in the phantom were uniformly distributed but the amplitude of the backscattered ultrasound from each scatterer was set to unity. Furthermore, to avoid clustering, the placement of the point scatterers was limited to a minimum distance of 50 μm from each other.

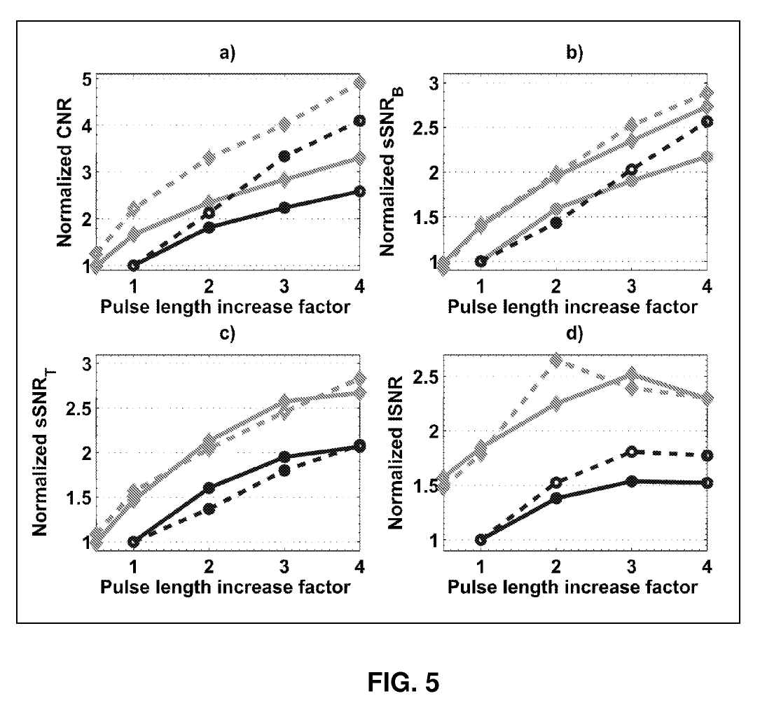

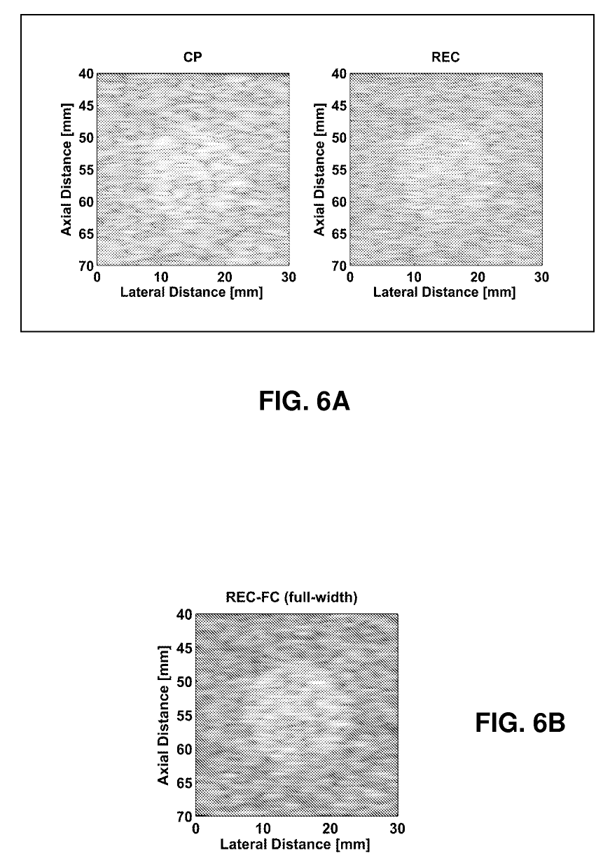

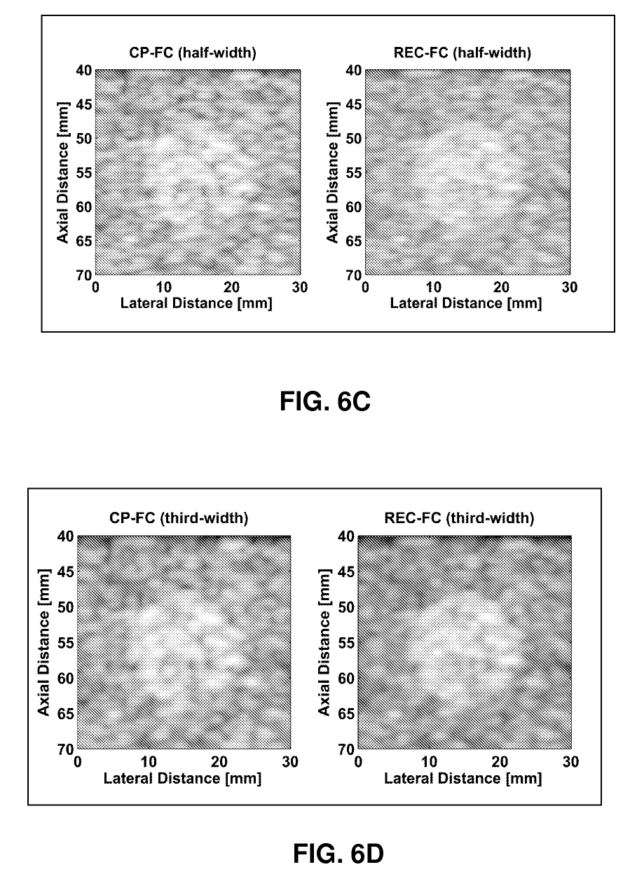

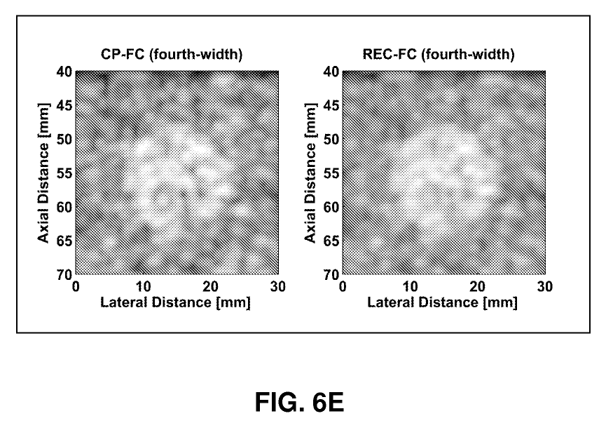

A total of 50 phantoms were simulated and evaluated with the image quality metrics discussed in section II. Attenuation and noise were not modeled in the simulations in order to examine the relationship of FC to speckle effect only. In simulations, no optimization of the γ parameter in the Wiener filter (Eq. 2) was used. However, in this study the γ parameter was determined by adjusting the parameter until it forced the Wiener filter towards an inverse filter in order to increase the axial resolution but not far enough that it would increase the noise above the background speckle level. A description of the filter banks designed along with the resolution needed to calculate lSNR in Eq. 9 are shown in Table I. Moreover, the CNR, sSNR, HPI, HO, and lSNR results obtained for all four cases (full-, half-, third-, and fourth-width) in addition to the reference scans (CP and REC with no compounding) are summarized in Table II. Further, the improvements in terms of CNR, sSNR_{B}, and sSNR_{T} are shown in FIG. 5(a,b,c). The B-mode images representing these results are displayed in FIG. 6.

Examination of the reference scans in FIG. 6(a) reveals that by using the REC technique the speckle size is finer when compared to CP. This smaller speckle size obtained by using REC means that the object boundaries would be more defined because there is more detail compared to CP. This resolution boost was then traded away to improve contrast by applying FC with subbands that have the same bandwidth as CP (full-width) as shown in FIG. 6(b). For the full-width case, the CNR, sSNR_{B}, and sSNR_{T} improved by a factor of 66%, 40%, and 47% when compared to CP. Essentially, a gain of CNR and sSNR was achieved while maintaining the same resolution that would be obtained by using CP methods. Examination of FIG. 5(d) suggests that by applying REC the target was more detectable than using CP methods. The lSNR for REC was increased by a factor of 57% when compared to CP. This would imply that the same lSNR value would be obtained for both CP and REC if a target of approximately 6.5 mm in diameter was imaged with REC rather than 15 mm as in CP.

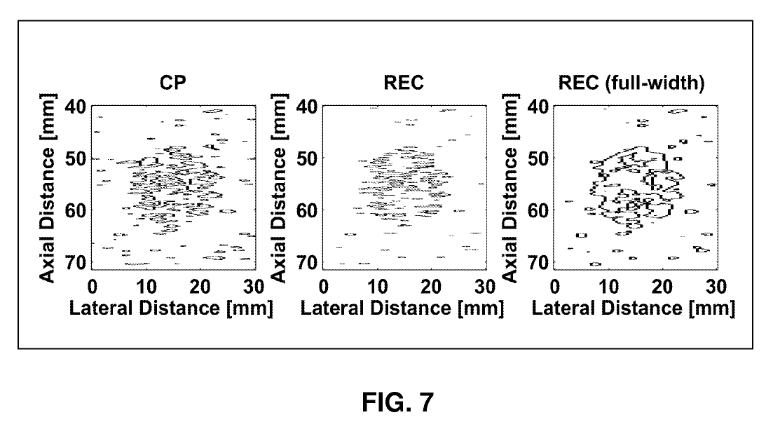

In addition to improvements in contrast, the boundaries of the target were more pronounced with REC-FC when compared to CP. This edge enhancement was due to the resolution enhancement in the REC reference image which led to an increased amount of detail as shown in FIG. 7. The images generated in FIG. 7 were generated by applying a −6-dB threshold on the envelopes of CP, REC, and REC-FC (full-width) and then estimating the margin strength. The difference in contrast between the background and the target was 6-dB; therefore, a threshold of −6-dB was considered. A threshold was applied to segment the target from the background, i.e. pixel values greater than −6-dB are considered part of the target and were assigned a value of one while all other pixel values were assigned a value of zero. Margin strength was then estimated to detect the strength of the distinct boundaries generated by applying the −6-dB threshold. Margin strength is defined by the following equation:

$$M S = E \{ \sqrt{{( \frac{d ROI}{d x} )}^{2} + {( \frac{d ROI}{d y} )}^{2}} \} \qquad ( 14 )$$

Where ROI is the region-of-interest within the envelope. From estimates of the margin strength, shown in FIG. 7, the target's edge was clearly defined and outlined for REC-FC when compared to CP. For REC this was not the case; however, when carefully examining REC versus CP, it was evident that REC had less background content around the target edges when compared to CP, allowing the boundaries to be traced.

The other cases evaluated were half-, third-, and fourth-width of CP impulse response, which would translate into a reduction by a factor of two, three and four in terms of axial resolution, respectively. The half-, third-, and fourth-width case FC filter banks was applied to both CP and REC as shown in FIG. 6(c-e). For these cases, unlike the full-width case where the axial resolution was restored to the original axial resolution with dramatic improvements in contrast, a tradeoff between the axial resolution and contrast resolution exists. In fact, it was observed from the lSNR values that the smaller the bandwidth of the subband used in FC, the loss in axial resolution outweighs the increase in contrast obtained. In the simulations, the lSNR for the fourth-width case when using REC-FC suffered from a significant drop, which is attributed to the deterioration of axial resolution; however, REC-FC was better than CP and CP-FC in terms of lSNR. These improvements in terms of lSNR quantified significant benefits of using REC-FC over CP-FC.

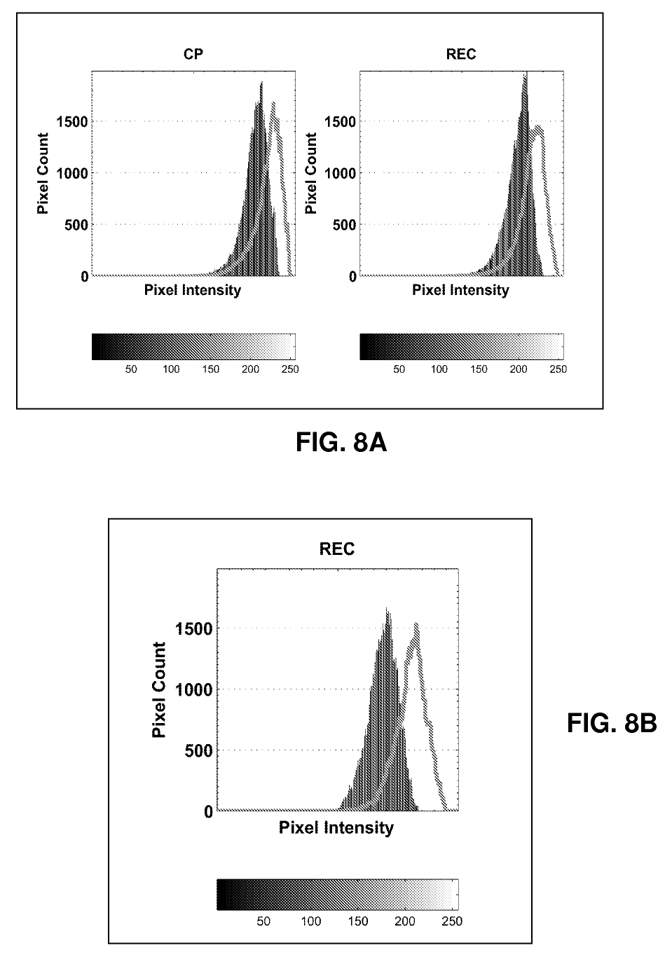

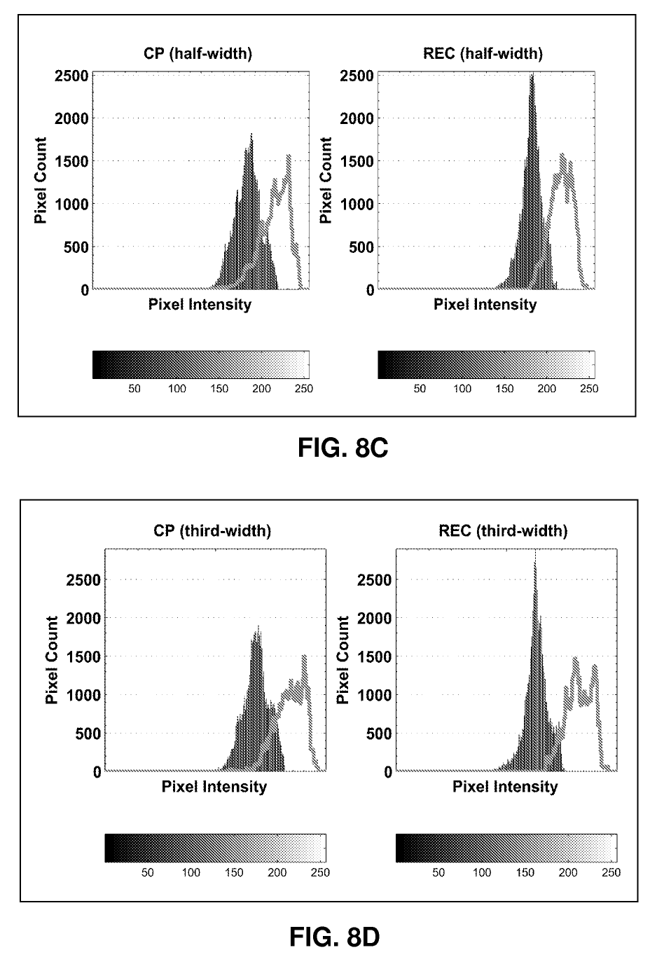

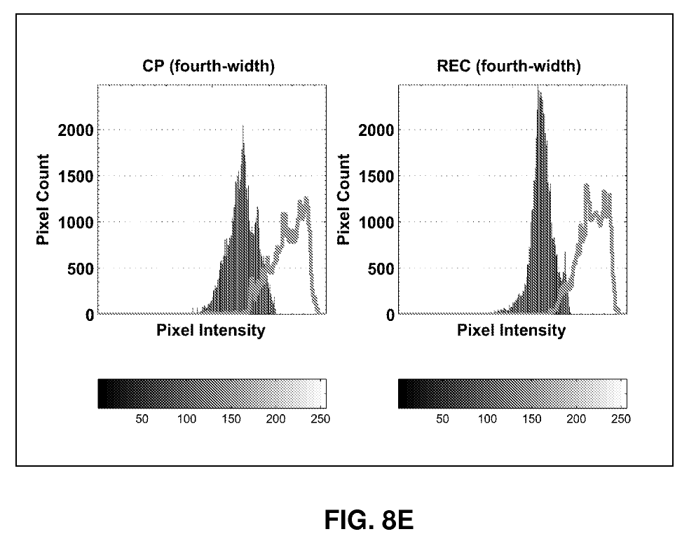

Histograms of the background and target regions for all four cases are shown in FIG. 8. From examination of the reference scans in FIG. 8(a) it is apparent that there is a large overlap in the distribution of grayscale pixel intensities between the background and the target, which was due to the large spread in the standard deviation about the mean pixel value of these regions. In fact, the overlap of these regions for the CP and REC reference scans were 26% and 25%, respectively. A large overlap between these two regions was undesirable because it would decrease the ability to detect the target from the background. By using FC, the overlap was reduced to 23% for the full-width case as shown in FIG. 8(b). This 3% decrease of overlap over CP was due to the mean pixel intensity shifting by +9.2 for the background region and +10.4 for the target region for the REC technique. More importantly this decrease in overlap can be attributed to a reduction of 10.7 and 11.3 in the standard deviation of pixel intensities about the mean for the background and the target regions, respectively. Moreover, as expected the percent overlap for the half-, third-, and fourth-width for CP-FC cases (FIGS. 8(c-e)) decreased; however, the overlap obtained with CP-FC was quite higher when compared to REC-FC.

Experiments were performed to validate the simulated results. A single-element weakly-focused (f/2.66) transducer (Panametrics, Waltham, Mass.) with a center frequency of 2.25 MHz and a 50% (at −3-dB) fractional bandwidth was used to image a phantom by translating the transducer laterally. There were two different experimental setups utilized; one for CP methods and another one for REC experiments. These setups would contain different noise levels due to the use of different excitation systems; therefore to avoid errors in the comparisons, the noise levels were normalized to an eSNR of 28 dB. Normalization of eSNR was accomplished by adding zero mean Gaussian white noise to the CP RF echo waveform. The two experimental setups are described as follows:

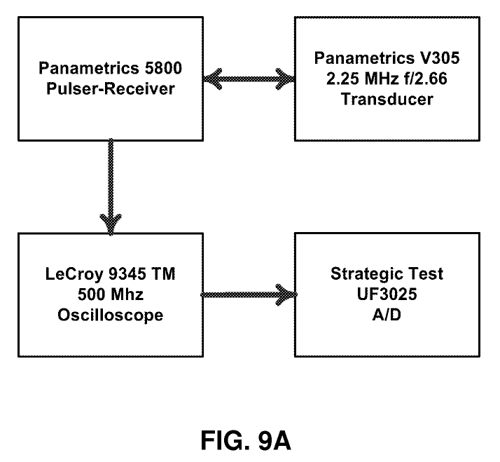

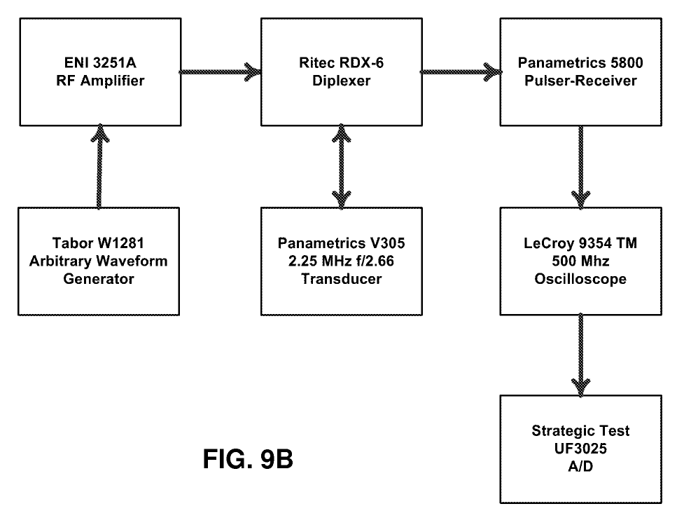

1. Conventional pulsing experimental setup: The transducer was excited by a pulser-receiver (Panametrics 5800, Waltham, Mass.) and the receive waveform was displayed on an oscilloscope (Lecroy 9354™, Chester Ridge, N.Y.) for visual verification. The echo signal was recorded at a rate of 100 MHz by a 12-bit A/D (Strategic Test Digitizing Board UF3025, Cambridge, Mass.) for further processing by a PC. A diagram of the experimental setup for CP is shown in FIG. 9(a).

2. REC experimental setup: The pre-enhanced chirp was generated in Matlab (The Mathworks Inc., Natick, Mass.) and downloaded to an arbitrary waveform generator (Tabor Electronics W1281A, Tel Hanan, Israel). The excitation signal was sampled at a rate of 100 MHz and amplified by an RF power amplifier (ENI 3251, Rochester, N.Y.). The amplified signal (50 dB) was connected to the transducer through a diplexer (Ritec RDX-6, Warwick, R.I.). The echo signal was received by a pulser-receiver (Panametrics 5800, Waltham, Mass.), which was displayed on an oscilloscope (Lecroy 9354™, Chester Ridge, N.Y.) for visual verification. The echo signal was recorded at a rate of 100 MHz by a 12-bit A/D (Strategic Test Digitizing Board UF3025, Cambridge, Mass.) for further processing by a PC. A diagram of the experimental setup for REC is shown in FIG. 9(b).

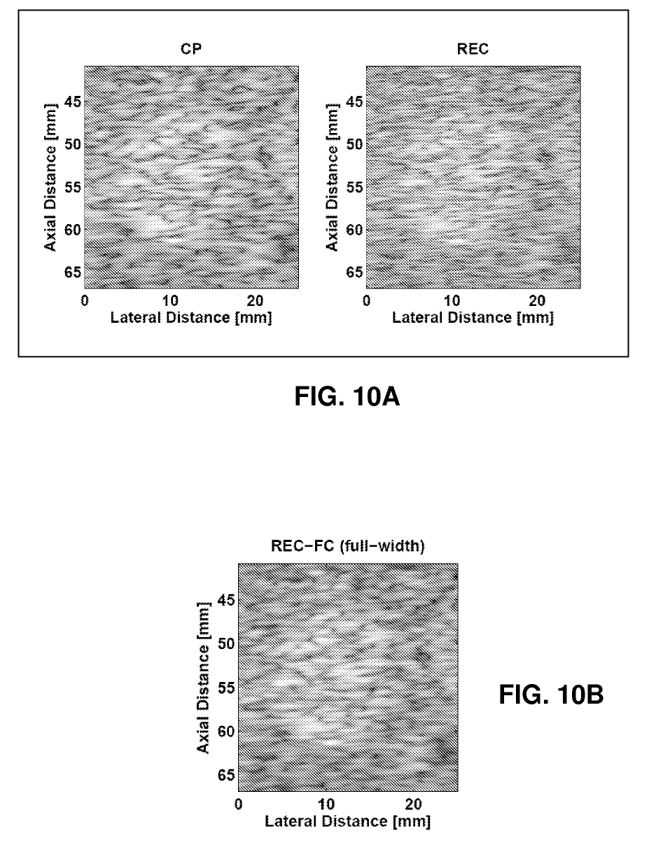

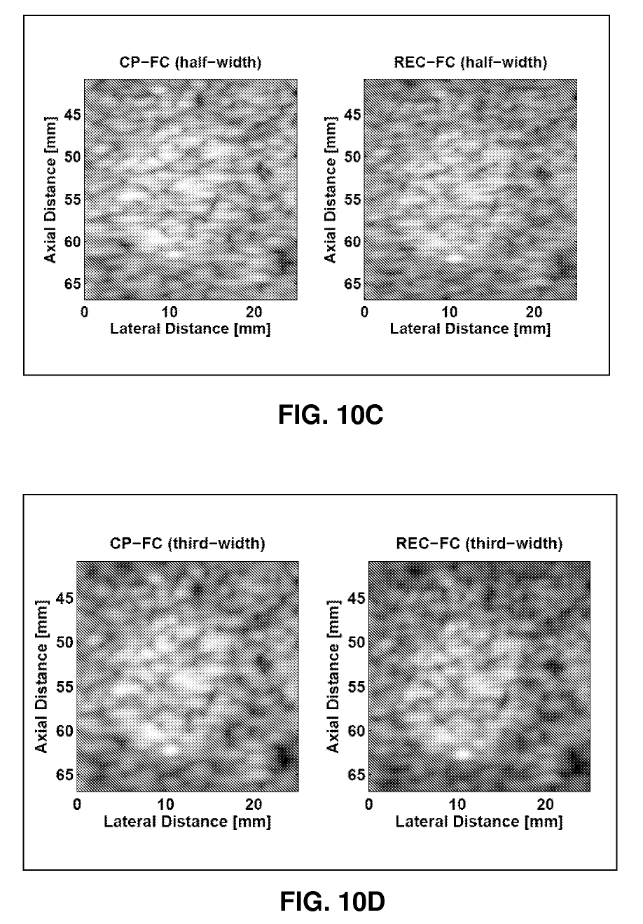

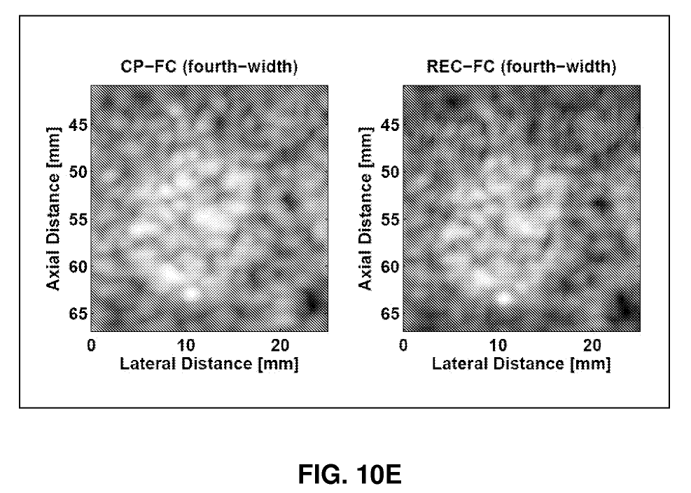

A tissue-mimicking phantom (ATS Laboratories Model 539, Bridgeport, Conn.) was used to assess the performance of REC-FC with the image quality metrics described in section II. The material from the tissue-mimicking phantom consisted of urethane rubber which has a speed of sound of 1450 m/s±1.0% at 23° C. and an attenuation coefficient of 0.5 dB/cm/MHz±5.0%. A +6-dB echogenic gray scale target structure with a 15-mm diameter at a depth of 4 cm was imaged for all four cases in addition to the reference signals. All measurements were conducted at room temperature in a tank of degassed water. Furthermore, in experimental measurements, no optimization of the γ parameter in the Wiener filter (Eq. 2) was used. Moreover, the same technique to determine the γ as described in section III was used for the experimental measurements. A description of the filter banks designed along with the resolution needed to calculate lSNR in Eq. 9 are shown in Table III. The CNR, sSNR, HPI, HO, and lSNR results obtained for all four cases (full-, half-, third-, and fourth-width) in addition to the reference scans (CP and REC with no compounding) are summarized in Table IV. Furthermore, the improvements in terms of CNR, sSNRB, and sSNRT are shown in FIG. 5(a,b,c). The B-mode images representing these results are displayed in FIG. 10.

Examination of the reference scans in FIG. 10(a) reveal that by using the REC technique the speckle size was finer when compared to CP, which was evident in the simulations. Next, FC was applied with subbands corresponding to the bandwidth of the original impulse response of the source under CP methods (full-width) to improve contrast as shown in FIG. 10(b). For the full-width case, the CNR, sSNR_{B} and sSNRT using REC-FC improved by a factor of 121%, 41%, and 57% when compared to CP. An increase in CNR and sSNR was obtained while maintaining the same axial resolution that would be obtained by using CP methods. Therefore, the same enhanced definition of object boundaries observed in simulated B-mode images and quantified through the margin strength when using REC leads to better definition of the object boundaries because there is more detail compared to CP, was observed in the experimental results.

Therefore, when applying REC-FC this extra information can enhance the results obtained when compared to CP. Evaluating lSNR results shown in FIG. 5 suggest that by using REC the lSNR improved by 48% over CP indicating improved target detectability. This would imply that the same lSNR value would be obtained for both CP and REC if a target of approximately 7.8 mm in diameter was imaged with REC rather than 15 mm as in CP.

A comparison of experimental results against simulations reveals the improvement for the full-width case in terms of sSNR_{B} and sSNR_{T} remained approximately the same, while the improvement in terms of CNR was almost doubled in the experiment (121%) when compared to simulations (66%). The next case evaluated was half-width of CP impulse response, which consisted of subbands that use half of the bandwidth of the impulse response of the source under CP methods. The half-width case was applied to both CP and REC as shown in FIG. 10(c). Comparing experimental results to simulations indicated that the improvements for the half-width case in terms of sSNR_{B} and sSNR_{T} were quite similar, while the improvement in terms of CNR was larger in the experiment (230%) when compared to simulations (134%). There are two other cases evaluated shown in FIGS. 10(d) and 10(e): third-width and fourth-width. These cases represent a pulse length increase factor of three and four, respectively. For the third- and fourth-width REC-FC cases, the lSNR dropped, but yet maintained higher values than CP, for the third-, and fourth-width case with increases of 139% and 130%, respectively. This decrease relates to the visible effects target edge blurring due to the resolution decrease in B-mode images. Therefore, based on simulation and experimental results it may not be recommended to drive REC-FC beyond the half-width case.

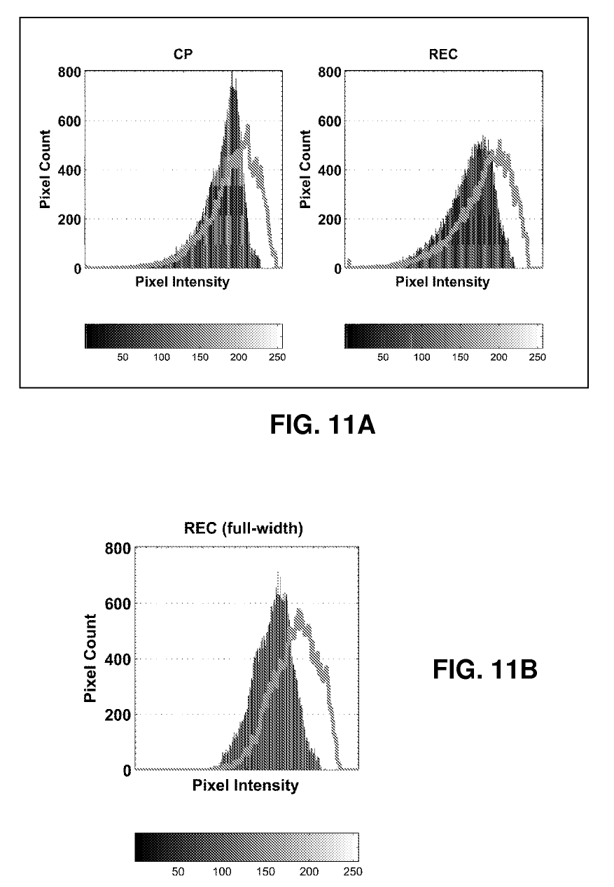

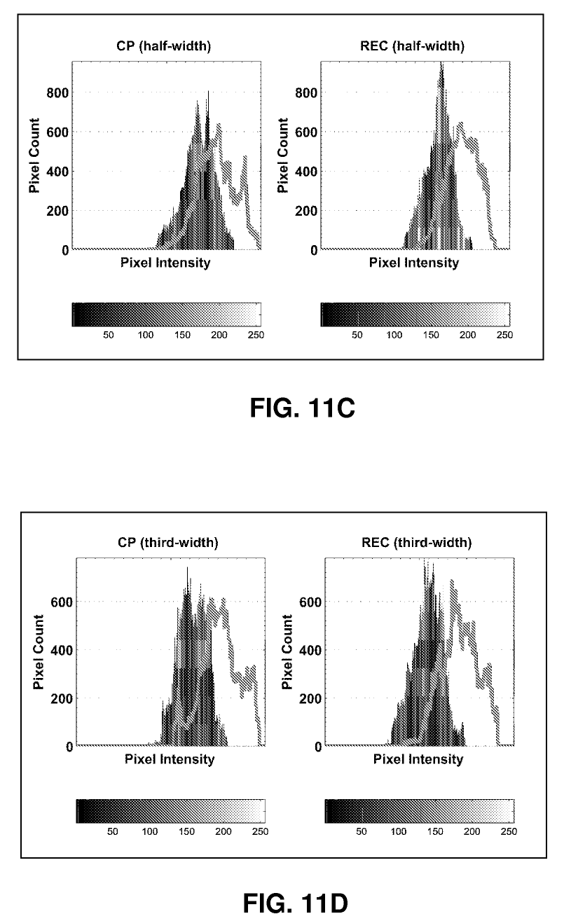

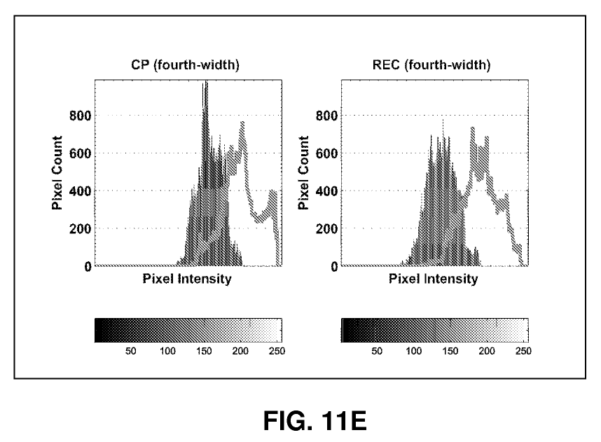

Histograms of the background and target region for all four cases are shown in FIG. 11. Examination of the reference scans shown in FIG. 11(a) reveal that there is a large overlap in the distribution of grayscale pixel intensities between the background and the target, which was due to the large spread in the standard deviation about the mean pixel intensity of these regions. In fact, the overlap of these regions for the CP and REC reference scan were both 35%. By using REC-FC, the overlap was reduced to 33% for the full-width case as shown in FIG. 11(b). A major difference between simulations and experiments was that the mean pixel intensity value for the background region increased during simulations while the value decreased for experimental measurements. The mean pixel intensity values for the target increased for both simulation results and experimental measurements. This change in the mean pixel value may help explain how the CNR results improved drastically during experiments compared to simulations. Another factor that may have led to the greater improvement in CNR for experimental measurements versus simulations is the fact that the simulations did not contain noise.

In using a coding technique and FC, noise may be further reduced leading to enhanced CNR over just the compounding effect alone. Furthermore, the overlap for the half-, third-, and fourth-width for CP-FC cases (FIG. 11(c,d,e)) decreased; however, the overlap obtained with CP-FC is quite higher when compared to REC-FC. Nonetheless, it should be noted that although the overlap between these two regions was reduced, in addition to the contrast improvements as each FC case was applied, the reduction in axial resolution was deteriorating image quality for the third- and fourth-width cases. This reduction in image quality could reduce the ability to detect small targets. This effect was not as noticeable in the simulation results except for the fourth-width case.

A pulse compression and coded excitation technique, REC, was used to double the axial resolution, which translated into an increase in system bandwidth. The speckle reduction technique known as FC utilized this larger available bandwidth to improve image contrast in ultrasonic B-mode images. FC partitions the useable bandwidth by using subbands that are smaller than the system bandwidth to improve image contrast and reduce speckle noise but at the expense of axial resolution. Therefore, the major objective of this study was to establish the benefits of doubling the axial resolution by utilizing REC and then applying FC to take advantage of the larger useable bandwidth to increase the number of subbands.

Simulations and experimental measurements were used to establish the usefulness of the REC-FC technique in enhancing image contrast and reducing speckle noise. Simulations and experimental measurements suggest that REC-FC was a useful tool to obtain substantial improvements in terms of image contrast and to enhance the boundaries between the target and the background speckle noise. CNR, sSNR_{B}, and sSNR_{T} were increased in both simulations and experiments for all cases. Also, in simulations and experiments, the overlap between the background and the target regions in the histograms was significantly reduced as the subbands got smaller.

From the foregoing descriptions, it would be evident to an artisan with ordinary skill in the art that the aforementioned embodiments can be modified, reduced, or enhanced without departing from the scope and spirit of the claims described below. Accordingly, the reader is directed to the claims for a fuller understanding of the breadth and scope of the present disclosure.

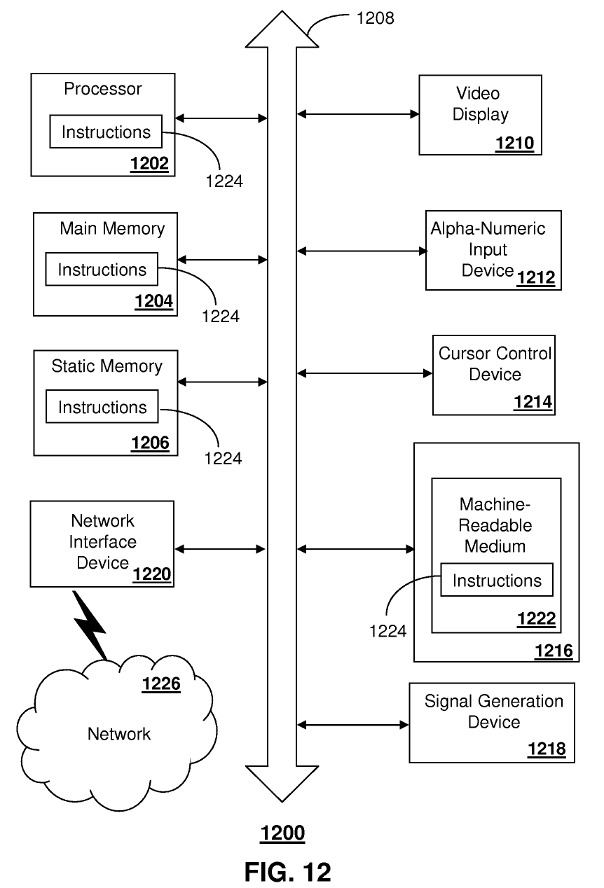

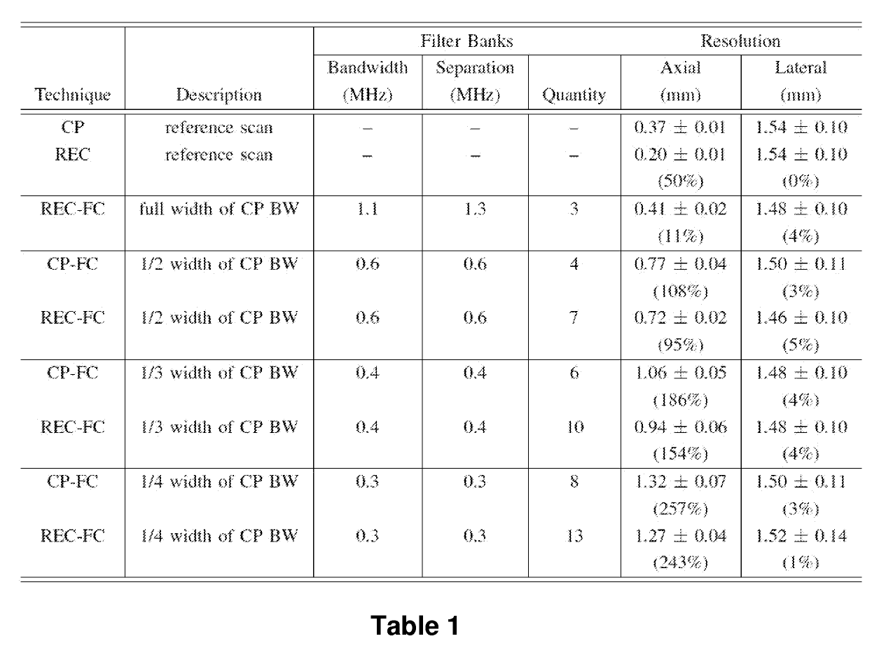

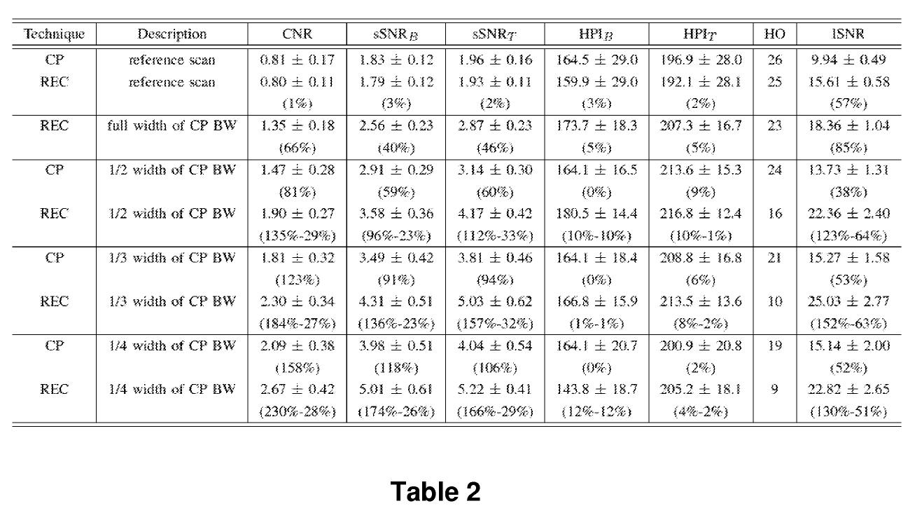

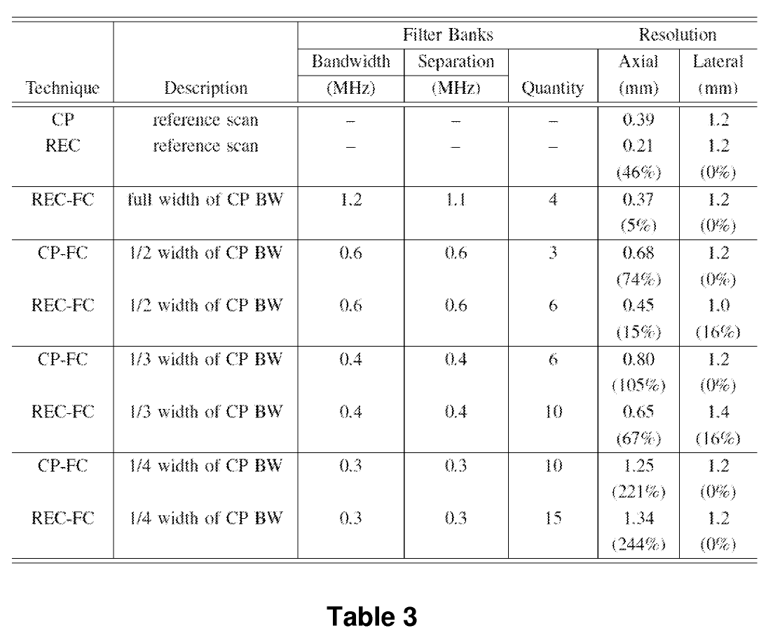

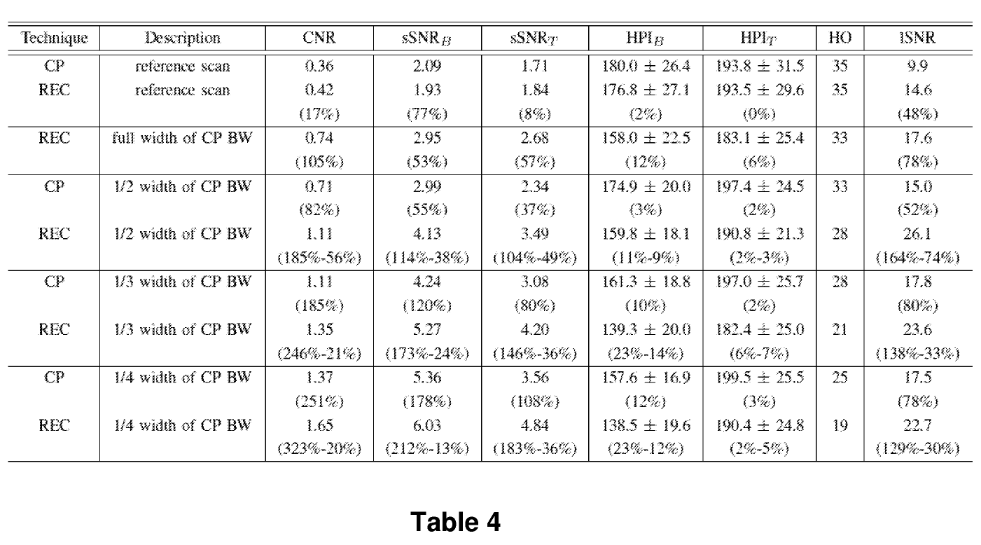

FIG. 12 depicts an exemplary diagrammatic representation of a machine in the form of a computer system 1200 within which a set of instructions, when executed, may cause the machine to perform any one or more of the methodologies discussed above. In some embodiments, the machine operates as a standalone device. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment.

The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. It will be understood that a device of the present disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methodologies discussed herein.

The computer system 1200 may include a processor 1202 (e.g., a central processing unit (CPU), a graphics processing unit (GPU, or both), a main memory 1204 and a static memory 1206, which communicate with each other via a bus 1208. The computer system 1200 may further include a video display unit 1210 (e.g., a liquid crystal display (LCD), a flat panel, a solid state display, or a cathode ray tube (CRT)). The computer system 1200 may include an input device 1212 (e.g., a keyboard), a cursor control device 1214 (e.g., a mouse), a disk drive unit 1216, a signal generation device 1218 (e.g., a speaker or remote control) and a network interface device 1220.

The disk drive unit 1216 may include a machine-readable medium 1222 on which is stored one or more sets of instructions (e.g., software 1224) embodying any one or more of the methodologies or functions described herein, including those methods illustrated above. The instructions 1224 may also reside, completely or at least partially, within the main memory 1204, the static memory 1206, and/or within the processor 1202 during execution thereof by the computer system 1200. The main memory 1204 and the processor 1202 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine readable medium containing instructions 1224, or that which receives and executes instructions 1224 from a propagated signal so that a device connected to a network environment 1226 can send or receive voice, video or data, and to communicate over the network 1226 using the instructions 1224. The instructions 1224 may further be transmitted or received over a network 1226 via the network interface device 1220.

While the machine-readable medium 1222 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure.

The term “machine-readable medium” shall accordingly be taken to include, but not be limited to: solid-state memories such as a memory card or other package that houses one or more read-only (non-volatile) memories, random access memories, or other re-writable (volatile) memories; magneto-optical or optical medium such as a disk or tape; and carrier wave signals such as a signal embodying computer instructions in a transmission medium; and/or a digital file attachment to e-mail or other self-contained information archive or set of archives is considered a distribution medium equivalent to a tangible storage medium. Accordingly, the disclosure is considered to include any one or more of a machine-readable medium or a distribution medium, as listed herein and including art-recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same functions are considered equivalents.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

### References

[1] J. G. Abbot, and F. L. Thurstone, “Acoustic speckle: theory and experimental analysis,” Ultrason. Imag., vol. 1, pp. 303-324, 1979.
[2] S. M. Gehlbach, and F. G. Sommer, “Frequency diversity speckle processing,” Ultrason. Imag., vol. 9, pp. 92-105, April 1987.
[3] P. A. Magnin, O. T. von Ramm, and F. L. Thurstone, “Frequency compounding for speckle contrast reduction in phased array images,” Ultrason. Imag., vol. 4, pp. 267-281, 1982.
[4] H. E. Melton, and P. A. Magnin, “A-Mode speckle reduction with compound frequencies and compound bandwidths,” Ultrason. Imag., vol. 6, pp. 159-173, 1984.
[5] V. L. Newhouse, N. M. Bilgutay, J. Saniie, and E. S. Furgason, “Flaw-to-grain echo enhancement by split-spectrum processing,” Ultrasonics, pp. 59-68, March 1982.
[6] G. E. Trahey, J. W. Allison, S. W. Smith, and O. T. von Ramm,, “A quantitative approach to speckle reduction via frequency compounding,” Ultrason. Imag., vol. 8, pp. 151-164, July 1986.
[7] F. L. Lizzi, M. E. Elbaum, and E. Feleppa, “Frequency diversity for image enhancement,” U.S. Pat. No. 4,561,019, Dec. 24, 1985.
[8] P. F. Stetson, F. G. Sommer, and A. Macovski, “Lesion contrast enhancement in medical ultrasound imaging,” IEEE Trans. Med. Imag., vol. 16, pp. 416-425, August 1997.
[9] M. L. Oelze, “Bandwidth and resolution enhancement through pulse compression,” IEEE Trans. Ultrason. Ferroelect. Freq. Contr., vol. 54, pp. 768-781, April 2007.
[10] J. K. Tsou, J. Liu, and M. F. Insana, “Modeling and phantom studies of ultrasonic wall shear rate measurements using coded excitation,” IEEE Trans. Ultrason. Ferroelect. Freq. Contr., vol. 53, pp. 724-734, 2006.

What is claimed is:

1. A method, comprising:
transmitting a coded ultrasound signal to an object;
receiving an altered coded ultrasound signal from the object;
decoding the altered coded ultrasound signal to attain a desired bandwidth that is larger than a bandwidth of an impulse response of an imaging system;
subdividing the attained bandwidth of the decoded ultrasound signal into a plurality of sub-bands; and
generating an image for each sub-band from signals included in each sub-band of the decoded ultrasound signal.

2. The method of claim 1, wherein the object comprises an anatomical object.

3. The method of claim 1, comprising creating the coded ultrasound signal from a nonlinear frequency modulated coded signal with a desired bandwidth and amplitude modulation having properties that augment the bandwidth of the imaging system.

4. The method of claim 1, comprising constructing a filter to reorient a phase of the altered coded ultrasound signal in order to compress a code embedded therein, thereby attaining the desired bandwidth upon reception.

5. The method of claim 1, wherein the decoding step preserves or enhances a spatial resolution compared to the impulse response of the imaging system.

6. The method of claim 1, comprising aggregating the image of each sub-band to create a composite image.

7. The method of claim 6, wherein the decoding step enhances a spatial resolution of the imaging system, and wherein the aggregating step enhances a contrast between objects depicted in the composite image.

8. The method of claim 6, creating the composite image from an average of the aggregated images.

9. The method of claim 6, wherein the composite image corresponds to an image of two or more dimensions.

10. The method of claim 1, wherein the coded ultrasound signal comprises frequency modulated codes with weighted amplitudes that shape a bandwidth of the coded ultrasound signal to a desired response.

11. The method of claim 1, wherein the coded ultrasound signal corresponds to digitally modulated codes.

12. The method of claim 1, wherein the altered coded ultrasound signal is a byproduct of scattering of the transmitted coded ultrasound signal in the object.

13. The method of claim 1, comprising comparing a code of the altered coded ultrasound signal to a base code used to form a filter to decode the altered coded ultrasound signal.

14. An ultrasonic (US) imaging system, comprising:
a transducer; and
a controller managing operations of the transducer, wherein the controller is operative to:
cause the transducer to transmit a coded ultrasound signal to an object;
cause the transducer to receive an altered coded ultrasound signal from the object;
decode the altered coded ultrasound signal to attain a desired bandwidth that exceeds an impulse response of the US imaging system;
subdivide the attained bandwidth of the decoded ultrasound signal into a plurality of sub-bands; and
generate an image for each sub-band from signals included in each sub-band of the decoded ultrasound signal.

15. The US imaging system of claim 14, wherein the object comprises an anatomical object, and wherein the transducer corresponds to a transducer array.

16. The US imaging system of claim 14, wherein the controller is operative to compress a code embedded in the altered coded ultrasound signal to attain the desired bandwidth.

17. The US imaging system of claim 14, wherein the controller is operative to combine the image of each sub-band to create a composite image.

18. The US imaging system of claim 17, wherein the controller is operative to create the composite image from an average of the combined images.

19. The US imaging system of claim 14, wherein the coded ultrasound signal corresponds to one among frequency modulated codes and digitally modulated codes.

20. A computer-readable storage medium, comprising computer instructions to:
decode a coded ultrasound signal to attain a desired bandwidth;
subdivide the attained bandwidth of the decoded ultrasound signal into a plurality of sub-bands; and
generate an image for each sub-band from signals included in each sub-band of the decoded ultrasound signal.

21. A computer-readable storage medium, comprising computer instructions to generate ultrasound images from a coded ultrasound signal processed according to a combination of a speckle reduction technique of frequency compounding and a coded excitation and pulse compression technique.

22. A method, comprising combining a speckle reduction technique of frequency compounding and a coded excitation and pulse compression technique to generate ultrasound images.

23. A method, comprising scanning an object with a Ultrasonic Imaging system operative to:
transmit a coded ultrasound signal to the object;
receive an altered coded ultrasound signal from the object;
decode the altered coded ultrasound signal to attain a desired bandwidth;
subdivide the attained bandwidth of the decoded ultrasound signal into a plurality of sub-bands; and
generate an image for each sub-band from signals included in each sub-band of the decoded ultrasound signal.

24. A method, comprising scanning an object with an Ultrasonic Imaging system that combines a speckle reduction technique of frequency compounding and a coded excitation and pulse compression technique to generate ultrasound images.

25. A method, comprising diagnosing a patient scanned with an Ultrasonic Imaging system that combines a speckle reduction technique of frequency compounding and a coded excitation and pulse compression technique to generate ultrasound images.

26. An Ultrasonic Imaging system, comprising a controller to generate ultrasound images by combining a speckle reduction technique of frequency compounding and a coded excitation and pulse compression technique.
